(19) United States
(12) Patent Application Publication
(10) Pub. No.: US 2018/0287426 A1
(43) Pub. Date: Oct. 4, 2018

(54) WIRELESS POWER TIME DIVISION TRANSMITTER AND COIL ARRAY

(71) Applicant: QUALCOMM Incorporated, San Diego, CA (US)

(72) Inventors: Mark White, II, San Diego, CA (US); Seong Heon Jeong, San Diego, CA (US); William Henry Von Novak, San Diego, CA (US)

(21) Appl. No.: 15/474,835

(22) Filed: Mar. 30, 2017

(51) Int. Cl. H02J 50/12; H02J 50/60; H02J 7/02; A61N 1/378

(52) U.S. Cl. CPC H02J 50/12; A61N 1/3787; H02J 7/025; H02J 50/60

(57) ABSTRACT

Systems and methods for wirelessly transferring power via magnetic field in a wireless power transfer system. A plurality of coils are placed at different locations around a body and configured to generate respective magnetic fields over different portions of the body to charge a chargeable device implanted within the body. A time division scheme is used such that no portion of the body experiences an average SAR over time that exceeds a designated SAR limit.

### BACKGROUND

### Technical Field

The present disclosure relates generally to wireless power transfer. More specifically, this disclosure relates to methods and apparatus for controlling wireless power transfer between power transfer units and power receiving units to provide wireless power to medical implants.

### Description of the Related Art

Medical “neuromodulation” implants are small devices that attach to nerves on humans or animals and may be used for monitoring or stimulation of nerves, allowing diagnosis and treatment of various diseases. In addition, other types of medical implants, such as insulin level monitors, insulin pumps, pacemakers, etc., may be used for a variety of other health diagnostic and treatment applications.

These different types of medical implants and devices all require power to operate. This power generally comes from a battery. However, due to being implanted inside an animal or human, it may be dangerous or risky to have to replace implant batteries regularly. Instead, it may be safer to recharge implant batteries wirelessly. However, wireless charging may be limited by safety concerns such as specific absorption rate (SAR), which may limit a field strength that may be transmitted, potentially reducing charging efficiency.

### SUMMARY

Various implementations of methods and devices within the scope of the appended claims each have several aspects, no single one of which is solely responsible for the desirable attributes described herein. Without limiting the scope of the appended claims, some prominent features are described herein.

An aspect of this disclosure is an apparatus for wirelessly transferring power via magnetic field in a wireless power transfer system. The apparatus comprises a first transmit circuit configured to generate a first magnetic field over a first portion of the living body towards a receiving circuit implanted inside the living body. The apparatus further comprises a second transmit configured to generate a second magnetic field over a second portion of the living body towards the receiving circuit. The apparatus further comprises a controller configured to operate the first transmit circuit at a first field strength over a first time period and the second transmit circuit at a second field strength over a second time period. The first and second transmit circuits are operated such that a collective strength of the generated first and second magnetic fields over a predetermined period of time remains below an average threshold value over all portions of the living body.

An aspect of this disclosure is a method for wirelessly transferring power via magnetic field in a wireless power transfer system. The method comprises, over a first time period, operating a first transmit circuit to generate a first magnetic field with a first field strength over a first portion of the living body towards a receiving circuit implanted inside the living body. The method further comprises, over a second time period, operating a second transmit configured to generate a second magnetic field at a second field strength over a second portion of the living body towards the receiving circuit. The first and second transmit circuits may be operated such that a collective strength of the generated first and second magnetic fields over a predetermined period of time remains below an average threshold value over all portions of the living body.

An aspect of this disclosure is an apparatus for wirelessly transferring power via magnetic field in a wireless power transfer system. The apparatus comprises first means for generating, over a first time period, a first magnetic field with a first field strength over a first portion of the living body towards a receiving circuit implanted inside the living body. The apparatus further comprises second means for generating, over a second time period, a second magnetic field at a second field strength over a second portion of the living body towards the receiving circuit. The first and second means are configured such that a collective strength of the generated first and second magnetic fields over a predetermined period of time remains below an average threshold value over all portions of the living body.

An aspect of this disclosure is a non-transitory computer readable medium. The non-transitory computer readable medium comprises code that, when executed, causes an apparatus to operate a first transmit circuit over a first time period to generate a first magnetic field with a first field strength over a first portion of the living body towards a receiving circuit implanted inside the living body. The non-transitory computer readable medium further comprises code that, when executed, causes the apparatus to operate a second transmit circuit over a second time period to generate a second magnetic field at a second field strength over a second portion of the living body towards the receiving circuit. The first and second transmit circuits may be operated such that a collective strength of the generated first and second magnetic fields over a predetermined period of time remains below an average threshold value over all portions of the living body

### BRIEF DESCRIPTION OF THE DRAWINGS

Details of one or more implementations of the subject matter described in this specification are set forth in the accompanying drawings and the description below. Other features, aspects, and advantages will become apparent from the description, the drawings, and the claims.

FIG. 1 is a functional block diagram of a wireless power transfer system, in accordance with one exemplary implementation.

FIG. 2 is a functional block diagram of a wireless power transfer system, in accordance with another exemplary implementation.

FIG. 3 is a schematic diagram of a portion of transmit circuitry or receive circuitry of FIG. 2 including a transmit or receive antenna, in accordance with exemplary implementations.

FIG. 4 is a functional block diagram of a transmitter that may be used in an inductive power transfer system, in accordance with exemplary implementations of the present disclosure.

FIG. 5 is a functional block diagram of a receiver that may be used in the inductive power transfer system, in accordance with exemplary implementations of the present disclosure.

FIG. 6 illustrates a transmitter that may be used to wireless transmit power to one or more medical implants (not shown) located within a body, in accordance with some embodiments.

FIG. 7 illustrates a diagram of transmitting coils arranged around a body containing a receiver, and the fields generated by the coils.

FIG. 8 illustrates a graph showing field strength against distance from transmitter.

FIG. 9 illustrates a voltage that may be received by a receiver implanted inside the body from a field generated by a transmitter, wherein the voltage received by the receiver may be relatively constant.

FIG. 10 illustrates a voltage that may be received by a receiver in response to a transmitter powered with a high power pulse and then shutting off for a period of time.

FIG. 11 illustrates field strengths of each coil at first location A within the body.

FIG. 12 illustrates field strengths of each coil at second location B within the body.

FIG. 13 illustrates field strengths of each coil at a receiver location within the body.

FIGS. 14A and 14B illustrates graphs showing generated field strengths of different coils and received voltage by a receiver over time, in accordance with some embodiments

FIG. 15 is a flowchart of an exemplary process for transmitted power via magnetic fields to a receiver of a medical implant implanted inside a body.

### DETAILED DESCRIPTION

The detailed description set forth below in connection with the appended drawings is intended as a description of exemplary implementations and is not intended to represent the only implementations in which the present disclosure may be practiced. The term “exemplary” used throughout this description means “serving as an example, instance, or illustration,” and should not necessarily be construed as preferred or advantageous over other exemplary implementations. The detailed description includes specified details for the purpose of providing a thorough understanding of the exemplary implementations. In some instances, some devices are shown in block diagram form.

Wirelessly transferring power may refer to transferring any form of energy associated with electric fields, magnetic fields, electromagnetic fields, or otherwise from a transmitter to a receiver without the use of physical electrical conductors (e.g., power may be transferred through free space). The power output into a wireless field (e.g., a magnetic field) may be received, captured by, or coupled by a “receiving coil” to achieve power transfer.

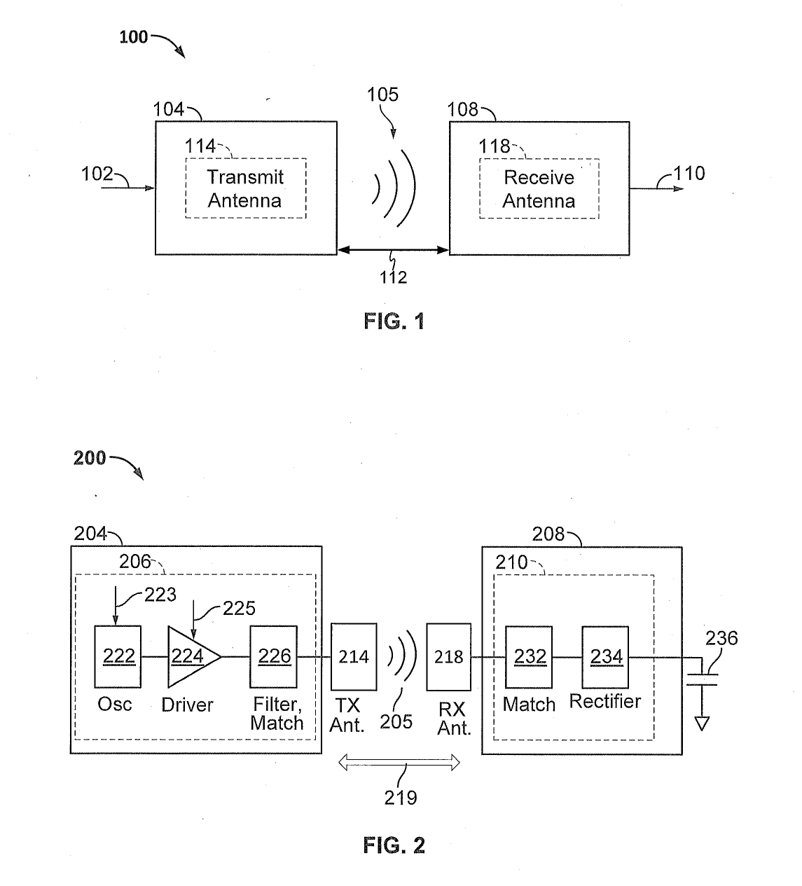

FIG. 1 is a functional block diagram of a wireless power transfer system 100, in accordance with one exemplary implementation. Input power 102 may be provided to a transmitter 104 from a power source (not shown) to generate a wireless (e.g., magnetic or electromagnetic) field 105 for performing wireless power transfer. A receiver 108 may couple to the wireless field 105 and generate output power 110 for storage or consumption by a device (not shown) coupled to the output power 110. Both the transmitter 104 and the receiver 108 are separated by a distance 112.

In one exemplary implementation, the transmitter 104 and the receiver 108 are configured according to a mutual resonant relationship. When the resonant frequency of the receiver 108 and the resonant frequency of the transmitter 104 are substantially the same or very close, transmission losses between the transmitter 104 and the receiver 108 are reduced. As such, wireless power transfer may be provided over a larger distance in contrast to purely inductive solutions that may require large antenna coils which are very close (e.g., sometimes within millimeters). Resonant inductive coupling techniques may thus allow for improved efficiency and power transfer over various distances and with a variety of inductive coil configurations.

The receiver 108 may receive power when the receiver 108 is located in the wireless field 105 produced by the transmitter 104. The wireless field 105 corresponds to a region where energy output by the transmitter 104 may be captured by the receiver 108. The wireless field 105 may correspond to the “near-field” of the transmitter 104 as will be further described below. The wireless field 105 may also operate over a longer distance than is considered “near field.” The transmitter 104 may include a transmit antenna 114 (e.g., a coil) for transmitting energy to the receiver 108. The receiver 108 may include a receive antenna or coil 118 for receiving or capturing energy transmitted from the transmitter 104. The near-field may correspond to a region in which there are strong reactance fields resulting from the currents and charges in the transmit antenna 114 that minimally radiate power away from the transmit antenna 114. The near-field may correspond to a region that is within about one wavelength (or a fraction thereof) of the transmit antenna 114.

FIG. 2 is a functional block diagram of a wireless power transfer system 200, in accordance with another exemplary implementation. The system 200 includes a transmitter 204 and a receiver 208. The transmitter 204 may include a transmit circuitry 206 that may include an oscillator 222, a driver circuit 224, and a filter and matching circuit 226. The oscillator 222 may be configured to generate a signal at a desired or target frequency that may be adjusted in response to a frequency control signal 223. The oscillator 222 may provide the oscillator signal to the driver circuit 224. The driver circuit 224 may be configured to drive the transmit antenna 214 at, for example, a resonant frequency of the transmit antenna 214 based on an input voltage signal (VD) 225. The driver circuit 224 may be a switching amplifier configured to receive a square wave from the oscillator 222 and output a sine wave. For example, the driver circuit 224 may be a class E amplifier.

The filter and matching circuit 226 may filter out harmonics or other unwanted frequencies and match the impedance of the transmitter 204 to the impedance of the transmit antenna 214. As a result of driving the transmit antenna 214, the transmit antenna 214 may generate a wireless field 205 to wirelessly output power at a level sufficient for charging a battery 236.

The receiver 208 may include a receive circuitry 210 that may include a matching circuit 232 and a rectifier circuit 234. The matching circuit 232 may match the impedance of the receive circuitry 210 to the receive antenna 218. The rectifier circuit 234 may generate a direct current (DC) power output from an alternate current (AC) power input to charge the battery 236, as shown in FIG. 2. The receiver 208 and the transmitter 204 may additionally communicate on a separate communication channel 219 (e.g., Bluetooth, ZigBee, cellular, etc.). The receiver 208 and the transmitter 204 may alternatively communicate via in-band signaling using characteristics of the wireless field 205.

The receiver 208 may be configured to determine whether an amount of power transmitted by the transmitter 204 and received by the receiver 208 is appropriate for charging the battery 236.

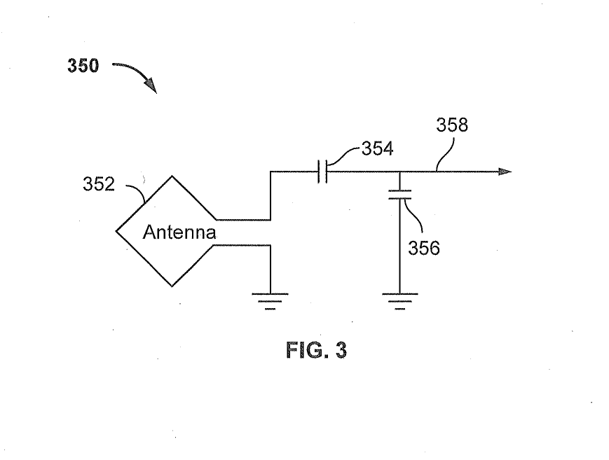

FIG. 3 is a schematic diagram of a portion of the transmit circuitry 206 or the receive circuitry 210 of FIG. 2 including a transmit or receive antenna, in accordance with exemplary implementations. As illustrated in FIG. 3, a transmit or receive circuitry 350 may include an antenna 352. The antenna 352 may also be referred to or be configured as a “loop” antenna 352. The antenna 352 may also be referred to herein or be configured as a “magnetic” antenna or an induction coil. The term “antenna” generally refers to a component that may wirelessly output or receive energy for coupling to another “antenna.” The antenna may also be referred to as a coil of a type that is configured to wirelessly output or receive power. As used herein, the antenna 352 is an example of a “power transfer component” of a type that is configured to wirelessly output and/or receive power.

The antenna 352 may include an air core or a physical core such as a ferrite core (not shown).

The transmit or receive circuitry 350 may form/include a resonant circuit. The resonant frequency of the loop or magnetic antennas is based on the inductance and capacitance. Inductance may be simply the inductance created by the antenna 352, whereas, capacitance may be added to the antenna's inductance to create a resonant structure at a desired or target resonant frequency. As a non-limiting example, a capacitor 354 and a capacitor 356 may be added to the transmit or receive circuitry 350 to create a resonant circuit. For a transmit circuitry, a signal 358 may be an input at a resonant frequency to cause the antenna 352 to generate a wireless field 105/205. For receive circuitry, the signal 358 may be an output to power or charge a load (not shown). For example, the load may comprise a wireless device configured to be charged by power received from the wireless field.

Other resonant circuits formed using other components are also possible. As another non-limiting example, a capacitor may be placed in parallel between the two terminals of the circuitry 350.

Referring to FIGS. 1 and 2, the transmitter 104/204 may output a time varying magnetic (or electromagnetic) field with a frequency corresponding to the resonant frequency of the transmit antenna 114/214. When the receiver 108/208 is within the wireless field 105/205, the time varying magnetic (or electromagnetic) field may induce a current in the receive antenna 118/218. As described above, if the receive antenna 118/218 is configured to resonate at the frequency of the transmit antenna 114/214, energy may be efficiently transferred. The AC signal induced in the receive antenna 118/218 may be rectified as described above to produce a DC signal that may be provided to charge or to power a load.

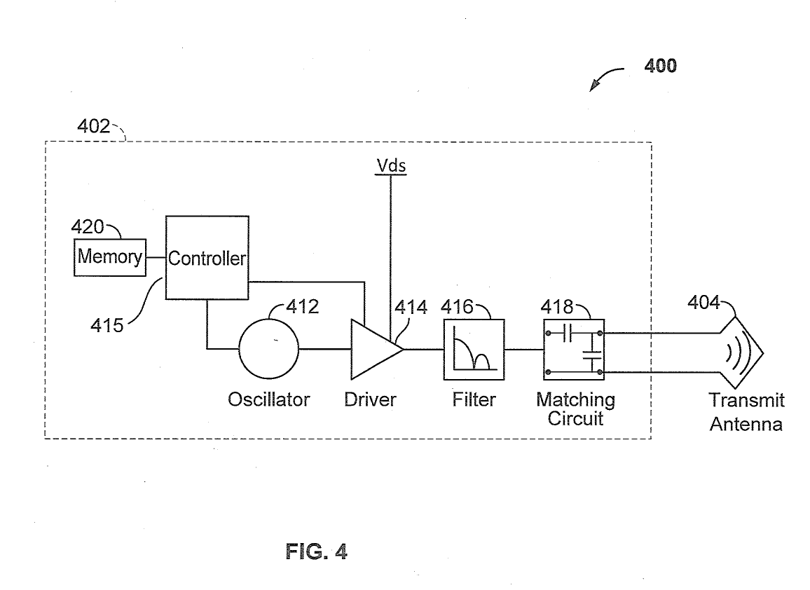

FIG. 4 is a simplified functional block diagram of a transmitter that may be used in an inductive power transfer system, in accordance with exemplary implementations of the present disclosure. As shown in FIG. 4, the transmitter 400 includes transmit circuitry 402 and a transmit antenna 404 operably coupled to the transmit circuitry 402. The transmit antenna 404 may be configured as the transmit antenna 214 as described above in reference to FIG. 2. In some implementations, the transmit antenna 404 may be a coil (e.g., an induction coil). In some implementations, the transmit antenna 404 may be associated with a larger structure, such as a table, mat, lamp, or other stationary configuration. The transmit antenna 404 may be configured to generate an electromagnetic or magnetic field. In an exemplary implementation, the transmit antenna 404 may be configured to transmit power to a receiver device within a charging region at a power level sufficient to charge or power the receiver device.

The transmit circuitry 402 may receive power through a number of power sources (not shown). The transmit circuitry 402 may include various components configured to drive the transmit antenna 404. In some exemplary implementations, the transmit circuitry 402 may be configured to adjust the transmission of wireless power based on the presence and constitution of the receiver devices as described herein. As such, the transmitter 400 may provide wireless power efficiently and safely.

The transmit circuitry 402 may further include a controller 415. In some implementations, the controller 415 may be a micro-controller. In other implementations, the controller 415 may be implemented as an application-specified integrated circuit (ASIC). The controller 415 may be operably connected, directly or indirectly, to each component of the transmit circuitry 402. The controller 415 may be further configured to receive information from each of the components of the transmit circuitry 402 and perform calculations based on the received information. The controller 415 may be configured to generate control signals for each of the components that may adjust the operation of that component. As such, the controller 415 may be configured to adjust the power transfer based on a result of the calculations performed by it.

The transmit circuitry 402 may further include a memory 420 operably connected to the controller 415. The memory 420 may comprise random-access memory (RAM), electrically erasable programmable read only memory (EEPROM), flash memory, or non-volatile RAM. The memory 420 may be configured to temporarily or permanently store data for use in read and write operations performed by the controller 415. For example, the memory 420 may be configured to store data generated as a result of the calculations of the controller 415. As such, the memory 420 allows the controller 415 to adjust the transmit circuitry 402 based on changes in the data over time.

The transmit circuitry 402 may further include an oscillator 412 operably connected to the controller 415. The oscillator 412 may be configured as the oscillator 222 as described above in reference to FIG. 2. The oscillator 412 may be configured to generate an oscillating signal (e.g., radio frequency (RF) signal) at the operating frequency of the wireless power transfer. In some exemplary implementations, the oscillator 412 may be configured to operate at the 6.78 MHz ISM frequency band. The controller 415 may be configured to selectively enable the oscillator 412 during a transmit phase (or duty cycle). The controller 415 may be further configured to adjust the frequency or a phase of the oscillator 412 which may reduce out-of-band emissions, especially when transitioning from one frequency to another. As described above, the transmit circuitry 402 may be configured to provide an amount of power to the transmit antenna 404, which may generate energy (e.g., magnetic flux) about the transmit antenna 404.

The transmit circuitry 402 may further include a driver circuit 414 operably connected to the controller 415 and the oscillator 412. The driver circuit 414 may be configured as the driver circuit 224 as described above in reference to FIG. 2. The driver circuit 414 may be configured to drive the signals received from the oscillator 412, as described above.

The transmit circuitry 402 may further include a low pass filter (LPF) 416 operably connected to the transmit antenna 404. The low pass filter 416 may be configured as the filter portion of the filter and matching circuit 226 as described above in reference to FIG. 2. In some exemplary implementations, the low pass filter 416 may be configured to receive and filter an analog signal of current and an analog signal of voltage generated by the driver circuit 414. The analog signal of current may comprise a time-varying current signal, while the analog signal of current may comprise a time-varying voltage signal. In some implementations, the low pass filter 416 may alter a phase of the analog signals. The low pass filter 416 may cause the same amount of phase change for both the current and the voltage, canceling out the changes. In some implementations, the controller 415 may be configured to compensate for the phase change caused by the low pass filter 416. The low pass filter 416 may be configured to reduce harmonic emissions to levels that may prevent self-jamming. Other exemplary implementations may include different filter topologies, such as notch filters that attenuate specified frequencies while passing others.

The transmit circuitry 402 may further include a fixed impedance matching circuit 418 operably connected to the low pass filter 416 and the transmit antenna 404. The matching circuit 418 may be configured as the matching portion of the filter and matching circuit 226 as described above in reference to FIG. 2. The matching circuit 418 may be configured to match the impedance of the transmit circuitry 402 (e.g., 50 ohms) to the transmit antenna 404. Other exemplary implementations may include an adaptive impedance match that may be varied based on measurable transmit metrics, such as the measured output power to the transmit antenna 404 or a DC current of the driver circuit 414. The transmit circuitry 402 may further comprise discrete devices, discrete circuits, and/or an integrated assembly of components.

Transmit antenna 404 may be implemented as an antenna strip with the thickness, width and metal type selected to keep resistance losses low.

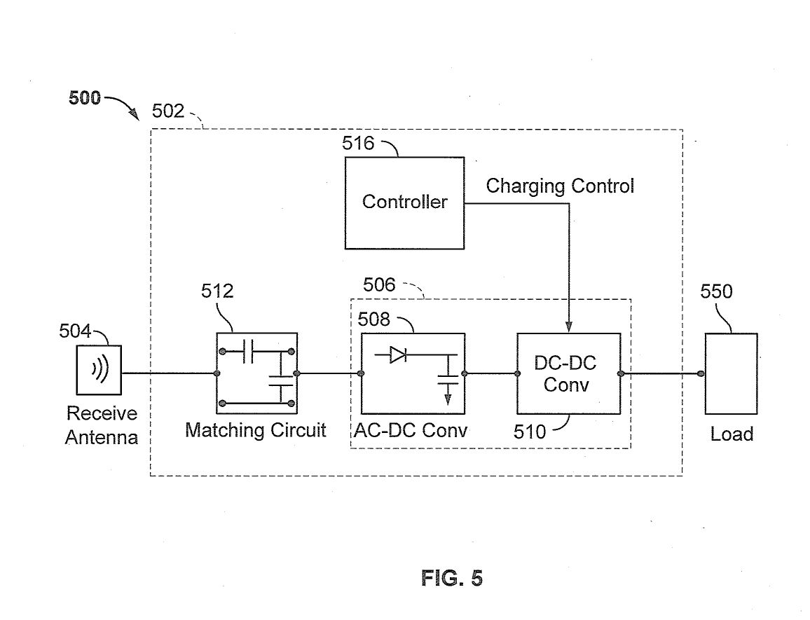

FIG. 5 is a block diagram of a receiver, in accordance with an implementation of the present disclosure. As shown in FIG. 5, a receiver 500 includes a receive circuitry 502, a receive antenna 504, and a load 550. The receiver 500 further couples to the load 550 for providing received power thereto. Receiver 500 is illustrated as being external to device acting as the load 550 but may be integrated into load 550. The receive antenna 504 may be operably connected to the receive circuitry 502. The receive antenna 504 may be configured as the receive antenna 218 as described above in reference to FIG. 2. In some implementations, the receive antenna 504 may be tuned to resonate at a frequency similar to a resonant frequency of the transmit antenna 404, or within a specified range of frequencies, as described above. The receive antenna 504 may be similarly dimensioned with transmit antenna 404 or may be differently sized based upon the dimensions of the load 550. The receive antenna 504 may be configured to couple to the magnetic field generated by the transmit antenna 404, as described above, and provide an amount of received energy to the receive circuitry 502 to power or charge the load 550.

The receive circuitry 502 may be operably coupled to the receive antenna 504 and the load 550. The receive circuitry may be configured as the receive circuitry 210 as described above in reference to FIG. 2. The receive circuitry 502 may be configured to match an impedance of the receive antenna 504, which may provide efficient reception of wireless power. The receive circuitry 502 may be configured to generate power based on the energy received from the receive antenna 504. The receive circuitry 502 may be configured to provide the generated power to the load 550. In some implementations, the receiver 500 may be configured to transmit a signal to the transmitter 400 indicating an amount of power received from the transmitter 400.

The receive circuitry 502 may include a processor-signaling controller 516 configured to coordinate the processes of the receiver 500 described below.

The receive circuitry 502 provides an impedance match to the receive antenna 504. The receive circuitry 502 includes power conversion circuitry 506 for converting a received energy into charging power for use by the load 550. The power conversion circuitry 506 includes an AC-to-DC converter 508 coupled to a DC-to-DC converter 510. The AC-to-DC converter 508 rectifies the AC energy signal received at the receive antenna 504 into a non-alternating power while the DC-to-DC converter 510 converts the rectified AC energy signal into an energy potential (e.g., voltage) that is compatible with the load 550. Various AC-to-DC converters are contemplated including partial and full rectifiers, regulators, bridges, doublers, as well as linear and switching converters.

The receive circuitry 502 may further include a matching circuit 512. The matching circuit 512 may comprise one or more resonant capacitors in either a shunt or a series configuration. In some implementations these resonant capacitors may tune the receive antenna to a specific frequency or to a specific frequency range (e.g., a resonant frequency).

The load 550 may be operably connected to the receive circuitry 502. The load 550 may be configured as the battery 236 as described above in reference to FIG. 2. In some implementations the load 550 may be external to the receive circuitry 502. In other implementations the load 550 may be integrated into the receive circuitry 502.

### Medical Implants and SAR Limitations

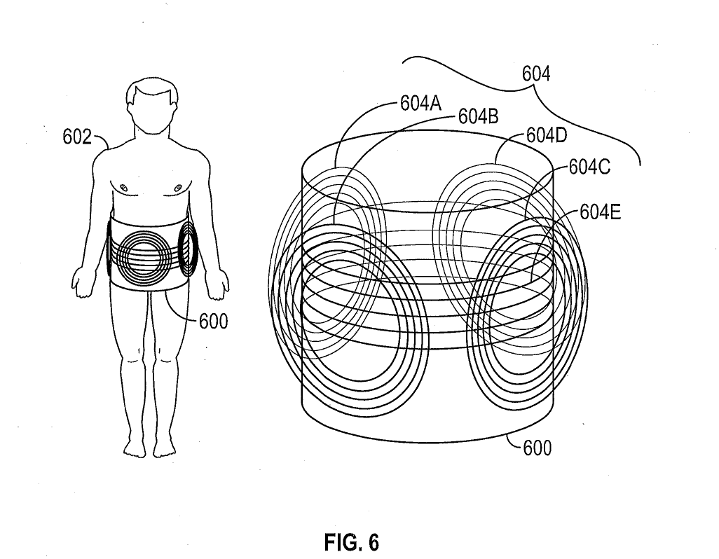

In some embodiments, wireless charging may be used to charge a medical implant implanted within the body of a human or animal. FIG. 6 illustrates a transmitter 600 located in proximity to a body 602 that may be used to wirelessly transmit power to one or more medical implants (not shown) located within the body 602, in accordance with some embodiments. The transmitter 600 may correspond to the transmitter 400 as illustrated in FIG. 4. The transmitter 600 comprises a plurality of coils 604 arranged around the body 602. For example, the coils 604 may comprise four coils (comprising coils 604A, 604B, 604C, and 604D) arranged at the front/back and left/right of the body 602. The coils 604 may further comprise one or more circumferential coils 604E arranged circumferentially around the body 602. In some embodiments, the coils 604A-E may correspond to the transmit antenna 404 as illustrated in FIG. 4. In some embodiments, different numbers and types of coils 604 may be used. For example, in some embodiments, the coils 604 may comprise one or more Helmholtz coil pairs, wherein the coils of each Helmholtz coil pair may be arranged on opposite sides of the body 602 (e.g., coils 604A and 604C may comprise a first Helmholtz coil pair, while coils 604B and 604D comprise a second Helmholtz coil pair). Each Helmholtz coil pair may be configured to operate at a particular frequency (e.g., 100 MHz).

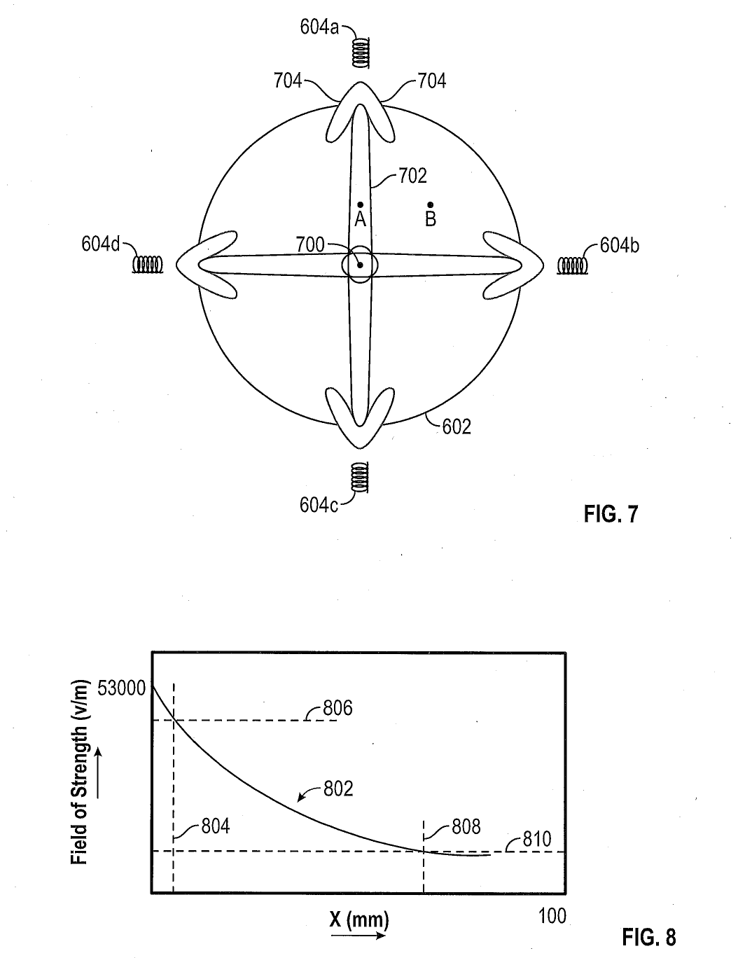

FIG. 7 illustrates a 2-D cross-sectional view of the body 602, where the coils 604A-D are arranged around the body 602, which contains an implanted receiver 700. The receiver 700 may correspond to the receiver 500 as illustrated in FIG. 5, and may be electrically connected via a wired connection to a chargeable device such as a medical implant (not shown) implanted within the body 602, e.g., a neuromodulation implant, an insulin pump, a pacemaker, etc.

Each of the coils 604A-D may be configured to generate a field having a directional transmission pattern, such that the field generated by each of the coils 604A-D extends over a particular area. For example, the coils 604A-D may each correspond to a high frequency directional antenna configured to operate at a frequency in the range of 1 GHz. In some embodiments, the distribution of the field generated by the coil 604A in 2-D space may be represented by a primary lobe 702 and two smaller secondary lobes 704. As such, by generating one or more magnetic fields through coils 604A through 604D, the transmitter 600 located outside the body 602 may be able to wirelessly transmit power to the receiver 700 coupled to the medical implant within the body 602.

In some embodiments, the receiver 700, in response to exposure to a generated field (e.g., from any of coils 604A through 604D), may generate an output voltage at an input of a rectifier (e.g., an input of AC-to-DC converter 508, as illustrated in FIG. 5). The resulting output voltage of the rectifier may be used to charge a load (e.g., load 550), which may correspond to a medical implant.

In some embodiments, losses in magnetic field strength (e.g., due to dissipation, absorption by tissue, and/or the like) between the transmitter 600 located outside the body 602 and the receiver 700 located within the body 602 may limit the ability of transmitter 600 to wirelessly charge the load 550 via the receiver 700. For example, in some embodiments, the receiver 700 may generate a peak to peak voltage of 150 mV at the rectifier input (e.g., the input of AC-to-DC converter 508) in response to the transmitted field from the transmitter 600 (e.g., through one or more of the coils 604A through 604D). If the AC-to-DC converter 508 of receiver 700 uses two 60 mV rectifiers, the resulting output voltage that may be used to charge the load 550 (e.g., the medical implant) will be approximately only 30 mV. This represents a rectifier efficiency of approximately 20%, which may be too low to effectively charge the load 550. In addition, in some embodiments, the voltage induced at the rectifier input (e.g., the input of AC-to-DC converter 508) may be insufficient to fully forward bias a diode of the rectifier (not shown), preventing rectification and charging of the load 550.

In addition, the strength of the field generated by the transmitter 600 may be limited by a SAR (specific absorption rate) limit. SAR generally refers to a measure of how much power is dissipated as heat in a unit of tissue mass, and may be measured in units of W/kg. For safety reasons, there are regulations on the amount of SAR experienced by human beings. For example, the FCC has established a limit on SAR of 1.6 W/kg, averaged over 1 g of tissue. In many regulations, SAR is measured over time (e.g., over a 10 minute period, a 30 minute period, or the like). As used herein, the term “SAR limit” may refer to a maximum amount of SAR experienced by a portion of tissue over a period of time as defined by a regulation (e.g., FCC regulations). The term “average SAR threshold” may refer to a level of field strength corresponding to an instantaneous SAR level (e.g., 1.6 W/kg over a portion of the body 602) corresponding to an average of the SAR limit over the period of time.

Furthermore, in some embodiments, the body 602 may contain multiple different implants (not shown) at different locations that may require wireless charging. For example, each implant may have a different depth or orientation relative to the transmitter 600. This may cause different degrees of coupling between the coils 604 of the transmitter 600 and the receivers of different implants (e.g., a difference of 100×). It may not be possible to have a single coil (e.g., coil 604A) of the transmitter 600 able to effectively support a full range of depths and orientations presented by the receivers of different implants.

FIG. 8 illustrates a graph showing a vertical y-axis representing the field strength a coil of the coils 604 of the transmitter 600 (e.g., coil 604A) and the horizontal x-axis represents a distance X (in millimeters (mm)) from the coil 604A. The field strength can be expressed in volts per meter, or V/m, or amps per meter, or A/m, depending on whether the field is electrical or magnetic. In some embodiments, if the coils 604 are operated at low operating frequencies (i.e. near field), the magnetic field may dominate. On the other hand, if the coils 604 are operated at high operating frequencies (i.e. far field), both magnetic and electrical fields may be present and should be considered.

The distance X may correspond to a distance from the coil 604A in a direction of the primary lobe 702 of the generated field of the coil 604A. As the coil 604A transmits wireless power through the body 602 towards the receiver 700 located within the body 602, the strength of the field generated by the coil 604A represented by the curve 802 may fall off in free space by a factor of 1/X². The falloff in the strength of the field generated by the coil 604A may further be exacerbated by field absorption by tissues within the body 602.

As illustrated in FIG. 8, if the receiver 700 of the implant is at a location 804 near the surface of the body 602 where the transmitting coil 604A is located (e.g., X is small), the strength of the field received by the receiver 700 will may be at a first level 806. On the other hand, if the receiver 700 is implanted at a location 808 deeper within the body 602 and further away from the transmitting coil 604A (e.g., X is large), the strength of the field received by the receiver 700 may be at a second level 810 that is significantly lower than the first level 806. In some embodiments, the strength of the field at second level 810 may be too low to power the implant electrically connected to the receiver 700. For example, in an embodiment, the maximum strength of the electric field of the transmitting coil 604A may be E=53000 V/m when X=0 mm, and may be 0 V/m when X=100 mm.

The amount of power from a generated field that is dissipated in tissue is proportional to field strength. As such, SAR associated with a field generated by the coil 604A will generally be greatest near the coil 604A (e.g., at the surface of the body 602) (e.g., where X is small), while becoming progressively smaller as distance X from the coil 604A increases. Thus, in order to comply with a SAR limit, the field strength that can be generated by the coil 604A will be limited by the resulting SAR near the surface of the body 602 close to the coil 604A.

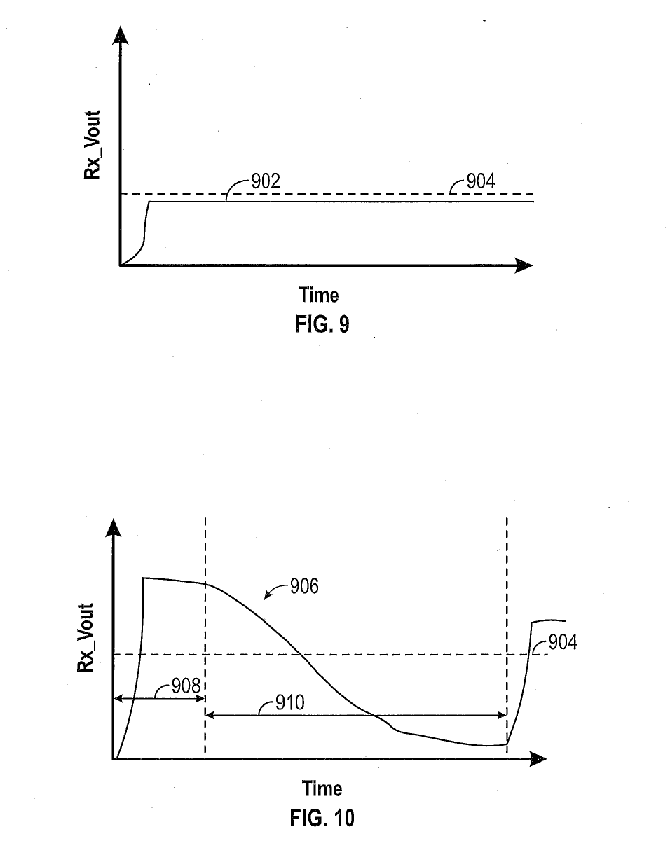

FIG. 9 and FIG. 10 show graphs having a vertical y-axis representing an output voltage (Rx_Vout) generated by the receiver 700 in response to a generated field by the coil 604A and a horizontal x-axis representing time. FIG. 9 illustrates a graph having a curve 902 illustrating the output voltage of the receiver 700 in response to the coil 604A generating a field of relatively constant strength over time. In some embodiments, in order to remain under a SAR limit, the strength of the field generated by the coil 604A may be limited such that the field strength near the surface of the body 602 does not exceed a threshold amount based upon the average SAR threshold. As such, the resulting output voltage of the receiver 700 may be at or below a level 904.

FIG. 10 illustrates a graph having a curve 906 illustrating the output voltage of the receiver 700 in response to the coil 604A generating a field having a strength that varies over time. For example, the transmitter 600 may inject a pulse into the coil 604A, such that the coil 604A alternately generates a field over a first period of time 908 and then shuts off (or generates a reduced field) over a second period of time 910. Because SAR may be measured over time, when the coil 604A is generating the field during the first time period 908, the strength of the field near the surface of the body 602 may exceed the average SAR threshold, as long as the average field strength over time remains under the average SAR threshold. As such, the field generated by the coil 604A during the first time period 908 may have a strength level greater than the strength of the field generated in relation to FIG. 9 (where the coil 604A is configured to generate a relatively constant field).

Due to the stronger field generated by the coil 604A during the first time period 908, the peak output voltage of the receiver 700 during the first time period 908 may be at a level 912 that exceeds the level 904. As such, the efficiency of the receiver 700 may potentially be increased. However, because the field generated by the coil 604A is then shut off or reduced over the second period of time 910, the average field strength over time at the surface of the body 602 near the coil 604A may remain under the average SAR threshold.

In some embodiments, the receiver 700 may contain a resonant circuit associated with a capacitance and an inductance defining one or more RC parameters. When the strength of the field generated by the coil 604A changes over time (e.g., between the first time period 908 and the second time period 910), the output voltage of the receiver 700, as illustrated by curve 906, may change based upon the one or more RC parameters. For example, the receiver 700 may output a high peak voltage as a result of a field generated from the coil 604A over the first time period 908, which may then decay in accordance with an RC time constant based upon the RC parameters of the receiver 700 over the second time period 910 (when the coil 604A is generating no field or a reduced field).

### Wireless Charging of Medical Implants Using Multiple Coils

In some embodiments, it is desirable to be able to maximize an amount of power that can be received by the receiver 700 while ensuring that the cumulative strength of the field(s) generated by the transmitter 600 does not exceed the SAR limit for any portion of the body 602. To do so, the transmitter 600 may transmit power through multiple coils 604 located over different parts of the body 602 (e.g., coils 604A through 604D, as illustrated in FIGS. 6 and 7). For example, referring back to FIG. 7, each of the coils 604A, 604B, 604C, and 604D may be configured to generate a field directed over a different portion of the body 602 (e.g., coil 604A may generate a field having a distribution represented as lobes 702 and 704 in FIG. 7). Because coils 604A-D are located over different portions of the body 602, the field strength experienced at different locations within the body 602 may vary, based upon which of coils 604A-D are closest to the location.

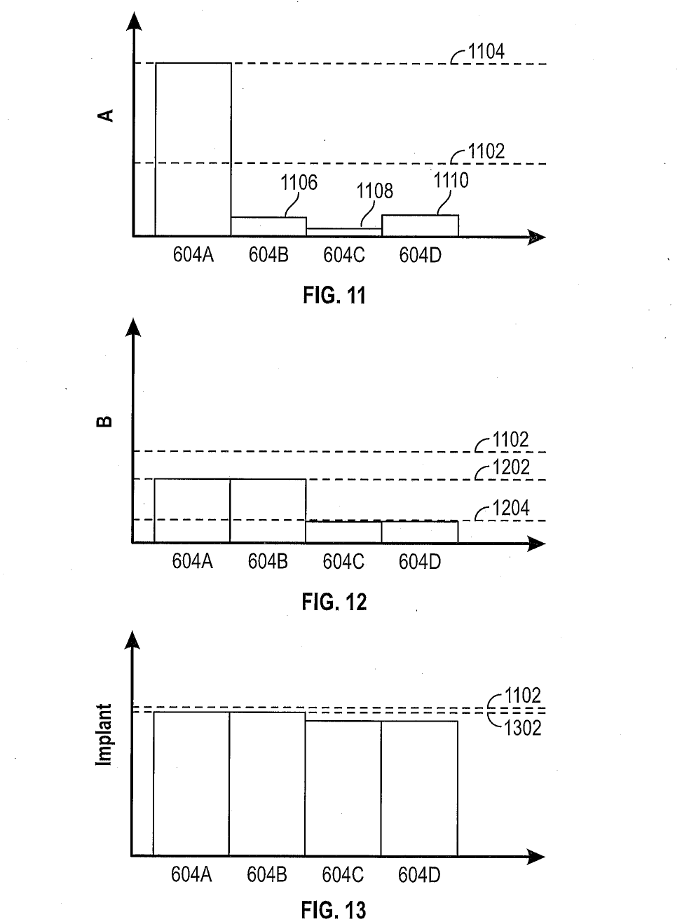

FIGS. 11-13 illustrated graphs showing the contribution of field strength from each of the coils 604A, 604B, 604C, and 604D at different locations within the body 602. FIG. 11 illustrates a graph showing the field strengths of each of coils 604A, 604B, 604C, and 604D at a first location A as illustrated in FIG. 7. FIG. 11 comprises a bar chart with field strength on the y-axis, and coils 604A, 604B, 604C, and 604D on the x-axis. Each bar of the graph of FIG. 11 depicts a contribution of field strength at location A from a respective coil 604A, 604B, 604C, or 604D. Because location A is located directly within the primary lobe 702 of the field generated by the coil 604A, the field strength from the coil 604A at location A may be at a level 1104 that is significantly higher than an average SAR threshold 1102. On the other hand, because location A is not near the field lobes generated by coils 604B, 604C, and 604D, field strength from coils 604B, 604C, and 604D at location A may be at levels 1106, 1108, and 1110 respectively, which are lower than the average SAR threshold 1102.

By limiting the duration that the field of the coil 604A is generated, the SAR experienced at location A with the body 602 may be kept below the average SAR threshold 1102. For example, in some embodiments, the coil 604A may be configured to alternately generate a field over a first time period (e.g., first time period 908), and then shut off or generate a field of reduced strength over a second time period (e.g., second time period 910). In some embodiments, coils 604B, 604C, and 604D may be configured to generate a field over different portions of the second time period 910. However because the strengths of the fields generated by coils 604B through 604D may have a strength at location A significantly below the average SAR threshold 1102, the average SAR level at location A may be kept below the average SAR threshold 1102 over the first and second time periods 908 and 910.

FIG. 12 illustrates a graph showing the field strengths of each of the coils 604A, 604B, 604C, and 604D at the second location B, as illustrated in FIG. 7. While location B is not located within a primary or secondary lobe of any of the fields generated by coils 604A through 604D (e.g., location B is outside the primary lobe 702 and secondary lobes 704 of the field generated by the coil 604A), the location B may still be exposed to portions of generated fields by coils 604A through 604D (e.g., via field leakage). Because location B is located closer to coils 604A and 604B than coils 604C and 604D, location B may experience a field from coils 604A and 604B at a level 1202 that is stronger than a field strength level 1204 from coils 604C and 604D. However, because none of the fields of coils 604A through 604D are directed towards location B, field strength levels 1202 and 1204 may both be below the average SAR limit 1102.

FIG. 13 illustrates a graph showing the field strengths of each of the coils 604A, 604B, 604C, and 604D at a location corresponding to the receiver 700, in accordance with some embodiments. In some embodiments, as illustrated in FIG. 7, the receiver 700 may be located within the primary field lobe of a plurality of the coils 604A through 604D (e.g., the location of receiver 700 is within the primary lobe 702 of the field generated by the coil 604A). Because the location of the receiver 700 is more distant from the coil 604A in comparison to location A, the peak field strength contributed by the coil 604A at the location of the receiver 700 may be at a level 1302 be lower than level 1102 (the field strength of coil 604A at location A). For example, the field strength level 1302 may be less than the average SAR threshold 1102. In some embodiments, the fields generated by coils 604B, 604C, and 604D may have strength levels at the location of the receiver 700 different from strength level 1302. For example, if the receiver 700 is located at a location further from the coil 604B compared to from the coil 604A, the strength level of the field generated by the coil 604B at the location of the receiver 700 may be lower than strength level 1302. In some embodiments, the coils 604A-604D may be configured to generate fields of strength level 1302 that are as close to the level 1102 as possible.

In addition, as illustrated in FIG. 7, the location of the receiver 700 is within the primary lobes of the fields generated by the coils 604B, 604C, and 604D. As such, the field strength contribution from the coils 604B, 604C, and 604D at the location of receiver 700 may also be at or near level 1302. In some embodiments, the coils 604A, 604B, 604C, and 604D may be configured such that only one of the coils 604A, 604B, 604C, and 604D generates a field at level 1302 at a given time (wherein the remaining coils are shut off or generate a reduced field). As such, the average field strength experienced at the location of the receiver 700 over time may at the level 1302 and below the average SAR threshold 1102.

### Time Division of Multiple Coils

As discussed above, in some embodiments, each of the coils 604A, 604B, 604C, and 604D of the transmitter 600 may be configured to generate a field during different time periods. The configuration of which of the coils 604A, 604B, 604C, and 604D generate their respective field over which times may be referred to as a “time division scheme.”

In some embodiments, by operating the coils 604A through 604D based upon a time division scheme, the receiver 700 implanted within the body 602 may be able to receive a more uniform cumulative field from the coils 604A through 604D. In addition, no location of the body 602 will exceed the SAR limit (e.g., be exposed to an average field strength over time that exceeds the average SAR threshold 1102). As illustrated in FIG. 7, the receiver 700 at the center of the body 602 may be able to receive wireless power via the primary lobes of the fields generated by each the coils 604A-D (e.g., primary lobe 702 of the field generated by the coil 604A). In some embodiments, the receiver 700 is able to receive power from all of the coils 604A through 604D, while in other embodiments, the receiver 700 may be able to receive power from some but not all of coils 604A through 604D.

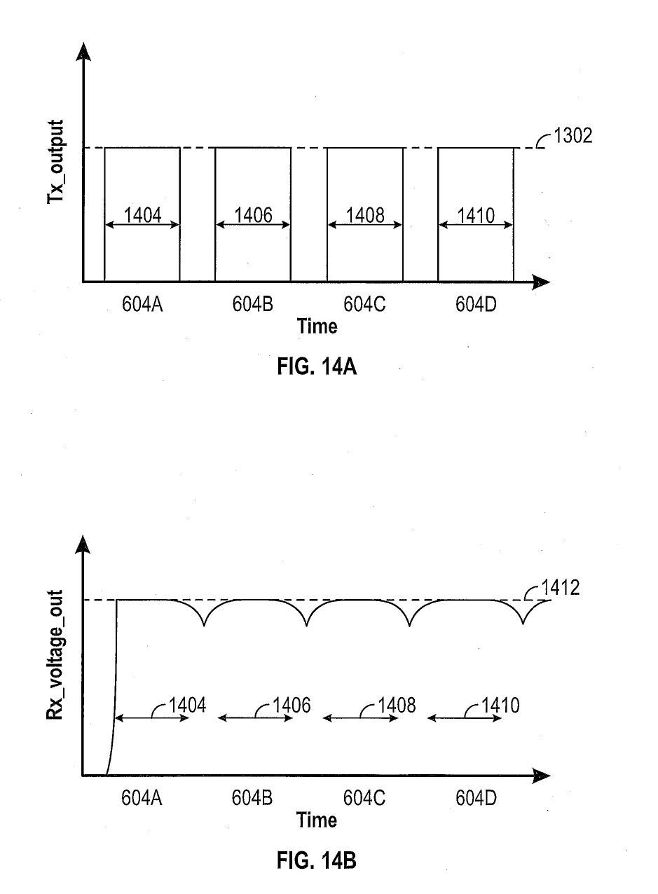

FIG. 14A illustrates a graph having a vertical y-axis representing generated field strengths from coils 604A through 604D on the y-axis, and a horizontal x-axis representing time, in accordance with some embodiments. In some embodiments, time division may be used to control the generation of fields from the coils 604A through 604D, such there is no overlap between the time periods during which each coil of coils 604A through D is generating a field (e.g., only one coil is generating a field at a time). For example, the coil 604A may be configured to generate a field having a strength level 1302 during a first time period 1404, during which coils 604B, 604C, and 604D are shut off or generate only a reduced field. Coils 604B, 604C, and 604D may be configured to generate fields having a strength of 1302 during second, third, and fourth time periods 1406, 1408, and 1410, respectively, during which the remain coils of coils 604A through 604D are shut down or generate only a reduced field. Because field strength level 1302 may be lower than the average SAR threshold 1102, the SAR at the location of the receiver 700 will not exceed the SAR limit. In some embodiments, by configuring the transmitter 600 such that only one coil of coils 604A through 604D is generating a field at a time, complexities that may arise due to phase construction/deconstruction between generated fields of two or more coils of coils 604A through 604D may be avoided.

As discussed above, each coil of the coils 604A through 604D may generate a field over a time period separate from those of each of the other coils. The fields generated by the coils 604A through 604D may have a strength level such that an average field strength over time will be below the average SAR threshold 1102 for all locations within the body 602. For example, while the field strength generated by the coil 604A may exceed the average SAR threshold 1102 at certain locations within the body 602 (e.g., at location A as illustrated in FIG. 7), because the coil 604A only generates a field during certain time periods (e.g., time period 1404) in accordance with a time division scheme, the average field strength at location A from the coils 604A through 604D over time may be below the average SAR threshold 1102.

In addition, the field generated by the coil 604A may have a strength level 1302 at the location of the receiver 700 that is below the average SAR threshold 1102. Because the receiver 700 may be at a location where it receives fields from different coils of the coils 604A through 604D during different time periods, the field strengths of each of the coils 604A through 604D may be configured such that the average field strength over time received at the location of the receiver 700 does not exceed the average SAR threshold 1102.

FIG. 14B illustrates a graph of having a vertical y-axis representing output voltage by the receiver 700 in response to fields generated by one or more coils of the coils 604A through 604D, and a horizontal x-axis representing time. Due to the RC parameters of the transmitter 700, or due to the resonant transmitter tank circuit “ringing down” when the driving signal is removed, the output voltage level may be at a level 1412 during time periods 1404, 1406, 1408, and 1410 when one of the coils 604A-604D is generating a field, and may decay between the time periods 1404, 1406, 1408, and 1410 (e.g., when none of the coils 604A-D are being actively driven). The rate of decay may be based upon an RC time constant of the receiver 700.

While FIG. 14A illustrates periods of time (e.g., between time periods 1404, 1406, 1408, and 1410) where none of the coils 604A-D are actively being driven, in some embodiments, the period of time between driving of successive coils 604A-D may be negligible. In addition, although FIGS. 14A and 14B illustrate the coils 604A-D generating fields over time periods in the order of coil 604A, coil 604B, coil 604C, and coil 604D, in other embodiments the coils 604A-D may generate fields in any order.

By having multiple coils 604A-604D arranged around and covering different areas of the body 602 and operated under a time division scheme (e.g., as illustrated in FIG. 14A), the SAR over any particular portion of the body may be kept under the SAR limit, while maximizing the amount of power that can be transmitted by the coils 604A-D. For example, by receiving a directed field from multiple coils of the coils 604A-604D, the average field strength received by the receiver 700 may be increased. On the other hand, areas near the surface of the body 602 (e.g., location A), where the field strength from each coil 604 is the strongest, may only receive a directed field from one of the coils 604A-604D. A time division scheme is implemented such that while the field strength of each of the coils 604A-D in regions closer to the surface of the body 602 may exceed the average SAR threshold during the period of time that the field is generated, the average field strength of each coil 604 over time remains under the average SAR threshold.

In some embodiments, the time division duration indicating a length of time each coil is operated for (e.g., a length of time period 1404, 1406, 1408, or 1410) may be based upon a charging requirement and a SAR measurement period. For example, if SAR is averaged over a 30 minute period, each time period 1404, 1406, 1408, and 1410 may correspond to a period of 5 to 20 minutes (e.g., 6 minutes). In some embodiments, the length of time periods 1404, 1406, 1408, and 1410 such that each of coils 604A through 604D will be operated to generate a respective field at least once during the SAR measurement period (e.g., the sum of time periods 1404, 1406, 1408, and 1410 is less or equal to the SAR measurement period). In some embodiments, the length of time periods 1404, 1406, 1408, and 1410 may be configured to be substantially the same, while in other embodiments they may be different (e.g., based upon a level of coupling between the receiver 700 and respective coils 604A through 604D)

In some embodiments, one or more circumferential coils (e.g., coil 604E, as illustrated in FIG. 6) may be used to transmit wireless power to the receiver 700, based upon an orientation of the receiver 700. For example, while the receiver 700 may be implanted into the body 602 having a particular orientation, the orientation of the receiver 700 may change or shift over time (e.g., as the body 602 moves). As such, the degree of coupling that can be achieved between the receiver 700 and each the coils 604A through 604E may change based upon the relative orientations of the receiver 700 and the coils 604A through 604E. For example, depending on orientation of receiver 700, it may be desirable to use transmit wireless power by generating a field using the circumferential coil 604E, in order to create a field in a direction that is in line with the receiver 700. In some embodiments, multiple circumferential coils may be used to transmit wireless power to the receiver 700.

In some embodiments, both circumferential coils (e.g., coil 604E) and Helmholtz coils (e.g., coils 604A, 604B, 604C, or 604D) may be used to generate magnetic fields for transmitting wireless power to the receiver 700. In some embodiments, the transmitter 600 may choose which of coils 604A through 604E to use for transmitting wireless power to the receiver 700, based upon a received voltage by the receiver 700. For example, the transmitter 600 may receive information from receiver 700 indicating a degree of coupling of the receiver 700 to one or more of the coil 604A through 604E, in order to determine which of the coils 604A through 604E to use for wireless charging.

While the above embodiments refer primarily to time divisions where only one coil is driven at a particular time, it is understood that in some embodiments, multiple coils of coils 604A through 604E may be driven at the same time. For example, in embodiments wherein at least some of the coils 604 are arranged in Helmholtz coil pairs (e.g., coils 604A and 604C comprising a first Helmholtz coil pair, and coils 604B and 604D comprising a second Helmholtz coil pair), the coils of each Helmholtz coil pair may be driven together to generate a substantially uniform magnetic field between the coils of the pair. In some embodiments, time division may be implemented such that only one Helmholtz coil pair of the coils 604 is driven at a particular time.

In some embodiments, Helmholtz coil pairs may be used to generate a substantially uniform field within a volume between the coils of the pair. However, the volume of the substantially uniform field generated by each Helmholtz coil pair may be limited (e.g., due to curvature in the coils causing the coils to deviate from “ideal” Helmholtz coils), and the field may become non-uniform in areas near the “corners” of the volume. In some embodiments, unexpected peaks in field strength may be present in areas near the coils of the Helmholtz pair. Through the use of time division schemes, the SAR in these areas due to the peaks in field strength may be kept under the SAR limit. In addition, in some embodiments, each Helmholtz coil pair may, due to curvatures in the coils of the pair, experience areas within the volume with null field, which may reduce an overall amount of SAR within the volume in the body 602.

In some embodiments, if a battery coupled to the implant and the receiver 700 is dead, it may be difficult for the transmitter 600 to determine which of the coils 604 to use, as the receiver 700 may be incapable of communicating. One approach to solving this is for the receiver 700 to send a load pulse to the transmitter 600 in response to receiving a DC voltage of some threshold. Whichever coil of coils 604A through 604E is able to trigger this load pulse with a lowest coil current may be designated as the coil 604 that couples best to the receiver 700. The transmitter 600 can then determine the optimal combination of coils of coils 604A through 604E and coil field strengths to optimize charging for each implant receiver 700.

It is understood that while the above embodiments describe an arrangement comprising four coils, the techniques described herein may be applied to a transmitter arrangement comprising any number of coils greater than 1.

### Process Flow

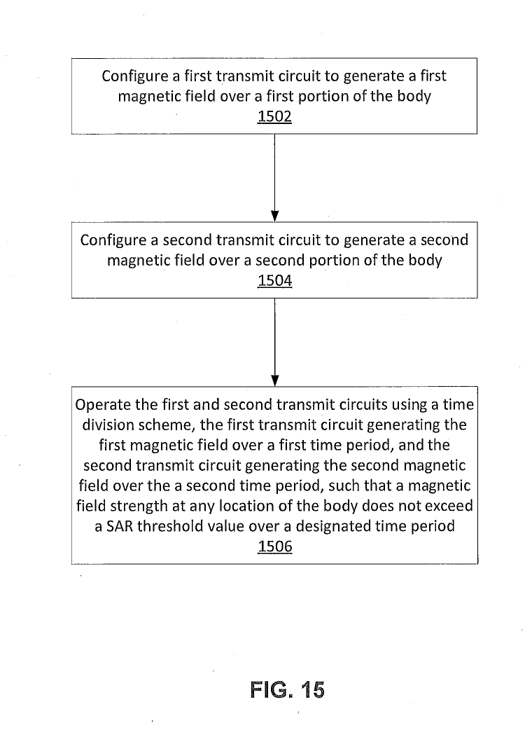

FIG. 15 is a flowchart of an exemplary process for transmitted power via magnetic fields to a receiver of a medical implant implanted inside a body. At block 1502, a first transmit circuit located outside the body is configured to generate a first magnetic field over a first portion of the body. At block 1504, a second transmit circuit is configured to generate a second magnetic field over a second portion of the body. In some embodiments, the first and second portions of the body may partially overlap. For example, the receiver of the medical implant may be located in an overlapping region of the first and second portions.

At block 1506, the first and second transmit circuits are operated using a time division scheme, such that the first transmit circuit generates the first magnetic field over a first time period, and the second transmit circuit generates the second magnetic field over the a second time period. In some embodiments, the first and second time periods are non-overlapping. The first and second time periods may be configured such that a magnetic field strength at any location of the body does not exceed a SAR threshold value over a designated time period. For example, for a location of the body near the first transmit coil but not near the second transmit coil, the strength of the magnetic field from the first transmit coil may exceed the SAR threshold value during the first time period, while the strength of the magnetic field from the second transmit coil during the second time period at the location may be below the SAR threshold value, such that the average field strength at the location over the first and second time periods is below the SAR threshold value.

For purposes of summarizing the disclosure, certain aspects, advantages and novel features of the present disclosures have been described herein. It is to be understood that not necessarily all such advantages can be achieved in accordance with any particular embodiment of the present disclosure. Thus, the present disclosure can be embodied or carried out in a manner that achieves or optimizes one advantage or group of advantages as taught herein without necessarily achieving other advantages as can be taught or suggested herein.

Various modifications of the above described embodiments will be readily apparent, and the generic principles defined herein can be applied to other embodiments without departing from the spirit or scope of the present disclosure. Thus, the present disclosure is not intended to be limited to the embodiments shown herein but is to be accorded the widest scope consistent with the principles and novel features disclosed herein.

What is claimed is:

1. An apparatus for wirelessly transferring power via magnetic field in a wireless power transfer system, the apparatus comprising:
a first transmit circuit configured to generate a first magnetic field over a first portion of the living body towards a receiving circuit implanted inside the living body,
a second transmit circuit configured to generate a second magnetic field over a second portion of the living body towards the receiving circuit; and
a controller configured to operate the first transmit circuit at a first field strength over a first time period and the second transmit circuit at a second field strength over a second time period, such that a collective strength of the generated first and second magnetic fields over a predetermined period of time remains below an average threshold value over all portions of the living body.

2. The apparatus of claim 1, wherein the average threshold value corresponds to a field strength associated with a specific absorption rate (SAR) limit.

3. The apparatus of claim 1, wherein the first transmit circuit and the second transmit circuit each comprise a Helmholtz coil pair.

4. The apparatus of claim 1, wherein the receiving circuit is electrically coupled to and configured to charge a battery of a medical implant implanted within the living body.

5. The apparatus of claim 1, wherein the first transmit circuit is configured to transfer wireless power to the receiving circuit during the first time period, and the second transmit circuit is configured to transfer wireless power to the receiving circuit during the second time period.

6. The apparatus of claim 1, wherein the first transmit circuit generates no magnetic field during the second time period, and the second transmit circuit generates no magnetic field during the first time period.

7. The apparatus of claim 1, wherein, at a first location within the first portion of the living body, a strength of the first magnetic field at the first location exceeds the average threshold value during the first time period, and a strength of the second magnetic field at the first location is below the average threshold value during the second time period, such that the average cumulative magnetic field strength at the first location from the first and second transmit circuits over the predetermined time period does not exceed the average threshold value.

8. The apparatus of claim 1, wherein the predetermined period of time is greater than a sum of the first and second time periods.

9. The apparatus of claim 1, wherein a strength of the first magnetic field decays with increasing distance from the first transmit circuit.

10. The apparatus of claim 1, wherein the first portion of the living body partially overlaps with the second portion of the living body.

11. A method for wirelessly transferring power via magnetic field in a wireless power transfer system, comprising:
over a first time period, operating a first transmit circuit to generate a first magnetic field with a first field strength over a first portion of the living body towards a receiving circuit implanted inside the living body; and
over a second time period, operating a second transmit circuit to generate a second magnetic field at a second field strength over a second portion of the living body towards the receiving circuit;
wherein a collective strength of the generated first and second magnetic fields over a predetermined period of time remains below an average threshold value over all portions of the living body.

12. The method of claim 11, wherein the average threshold value corresponds to a field strength associated with a specific absorption rate (SAR) limit.

13. The method of claim 11, wherein the first transmit circuit and the second transmit circuit each comprise a Helmholtz coil pair.

14. The method of claim 11, wherein the generated first and second magnetic fields are configured to charge a battery of a medical implant implanted, via the receiving circuit, within the living body.

15. The method of claim 11, wherein the first transmit circuit is configured to transfer wireless power to the receiving circuit during the first time period, and the second transmit circuit is configured to transfer wireless power to the receiving circuit during the second time period.

16. The method of claim 11, further comprising operating the first transmit circuit to generate no magnetic field during the second time period, and operating the second transmit circuit to generate no magnetic field during the first time period.

17. The method of claim 11, wherein the first and second transmit circuits are operated such that at a first location within the first portion of the living body, a strength of the first magnetic field at the first location exceeds the average threshold value during the first time period, and a strength of the second magnetic field at the first location is below the average threshold value during the second time period, such that the average cumulative magnetic field strength at the first location from the first and second transmit circuits over the predetermined time period does not exceed the average threshold value.

18. The method of claim 11, wherein the predetermined period of time is greater than a sum of the first and second time periods.

19. The method of claim 11, wherein a strength of the first magnetic field decays with increasing distance from the first transmit circuit.

20. The method of claim 11, wherein the first portion of the living body partially overlaps with the second portion of the living body.

21. An apparatus for wirelessly transferring power via magnetic field in a wireless power transfer system, the apparatus comprising:
first means for generating, over a first time period, a first magnetic field with a first field strength over a first portion of the living body towards a receiving circuit implanted inside the living body; and
second means for generating, over a second time period, a second magnetic field at a second field strength over a second portion of the living body towards the receiving circuit;
wherein a collective strength of the generated first and second magnetic fields over a predetermined period of time remains below an average threshold value over all portions of the living body.

22. The apparatus of claim 21, wherein the average threshold value corresponds to a field strength associated with a specific absorption rate (SAR) limit.

23. The apparatus of claim 21, wherein the first generating means is configured to transfer wireless power to the receiving circuit during the first time period, and the second generating means is configured to transfer wireless power to the receiving circuit during the second time period.

24. The apparatus of claim 21, wherein the first generating means generates no magnetic field during the second time period, and the second generating means generates no magnetic field during the first time period.

25. The apparatus of claim 21, wherein, at a first location within the first portion of the living body, a strength of the first magnetic field at the first location exceeds the average threshold value during the first time period, and a strength of the second magnetic field at the first location is below the average threshold value during the second time period, such that the average cumulative magnetic field strength at the first location from the first and second transmit circuits over the predetermined time period does not exceed the average threshold value.

26. A non-transitory computer readable medium comprising code that, when executed, causes an apparatus to:
operate a first transmit circuit over a first time period to generate a first magnetic field with a first field strength over a first portion of the living body towards a receiving circuit implanted inside the living body; and
operate a second transmit circuit over a second time period to generate a second magnetic field at a second field strength over a second portion of the living body towards the receiving circuit;
wherein a collective strength of the generated first and second magnetic fields over a predetermined period of time remains below an average threshold value over all portions of the living body.

27. The non-transitory computer readable medium of claim 26, wherein the average threshold value corresponds to a field strength associated with a specific absorption rate (SAR) limit.

28. The non-transitory computer readable medium of claim 26, wherein the first transmit circuit is configured to transfer wireless power to the receiving circuit during the first time period, and the second transmit circuit is configured to transfer wireless power to the receiving circuit during the second time period.

29. The non-transitory computer readable medium of claim 26, wherein the code, when executed, further causes the apparatus to operate the first transmit circuit to generate no magnetic field during the second time period, and operate the second transmit circuit to generate no magnetic field during the first time period.

30. The non-transitory computer readable medium of claim 26, wherein the first and second transmit circuits are operated such that at a first location within the first portion of the living body, a strength of the first magnetic field at the first location exceeds the average threshold value during the first time period, and a strength of the second magnetic field at the first location is below the average threshold value during the second time period, such that the average cumulative magnetic field strength at the first location from the first and second transmit circuits over the predetermined time period does not exceed the average threshold value.
